(19) United States
(12) Patent Application Publication
(10) Pub. No.: US 2020/0129737 A1
(43) Pub. Date: Apr. 30, 2020

(54) PICC Line Arm Sleeve

(71) Applicant: Shanita Woodard, Houston, TX (US)

(72) Inventor: Shanita Woodard, Houston, TX (US)

(21) Appl. No.: 16/012,762

(22) Filed: Jun. 20, 2018

(51) Int. Cl. A61M 25/02

(52) U.S. Cl. CPC A61M 25/02; A61M 2205/0205; A61M 2025/0213; A61M 2025/0206

(57) ABSTRACT

A peripherally inserted central catheter (PICC) line arm sleeve with a window-style opening design or slip-on structured design. The sleeve has treated mesh fabric, with properties including, but not limited to, anti-bacterial, odor-resistant, moisture-wicking, and breathability, to cover the PICC insertion area. Treated and non-treated solid fabric may be used in the body of the sleeve. Treated solid fabric used has the same properties as treated mesh fabric. Use of the sleeve is not limited but targets survivors undergoing treatment for cancer(s), infectious disease(s), and other medical issues that use PICC lines in the treatment plan.

### CROSS-REFERENCE TO RELATED APPLICATIONS

The present application claims priority to the earlier filed provisional applications having application No. 62/491,182 with EFS ID 29055873 and application no. 62492216 with EFS ID 29071275, and hereby incorporates subject matter of the provisional applications in their entirety.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable.

In the email dated Dec. 3, 2018, I requested an update on my application. I contacted the application assistance unit and understood I had been sent a letter that had an action required, but unfortunately, I did not have a copy of the letter and requested it be mailed again. I don't know if the letter was resent, but the next correspondence I did receive was the attached Abandonment/Termination Letter.

I did not intentionally not respond to the letter dated Jul. 9, 2018 and I am submitting this petition to request that my application be revived.

I have invested a lot in my product and will complete any required follow-ups to continue the review and examination of my patent application.

Thank you for your consideration.

This petition is submitted for U.S. nonprovisional patent application Ser. No. 16/012,762 submitted Jun. 20, 2018, with confirmation no. 2268, and EFS ID 32944702.

### BACKGROUND OF THE INVENTION

The invention described in the nonprovisional applications referenced in section [0001] is in the technical field of PICC, peripherally inserted central catheter, covers. More particularly, the invention is in the technical field of PICC, peripherally inserted central catheter, arm sleeves with anti-microbial, breathable mesh fabric over the PICC insertion area for survivors undergoing treatment for cancer(s), infectious disease(s), and other medical issues that use PICC lines in the treatment plan.

The invention is an arm sleeve designed to provide coverage for peripherally inserted central catheter lines (PICC) lines.

### STATEMENT UNINTENTIONAL DELAY

I submitted my provisional patent applications in June 2018. I have included 2 attachments of email exchanges with USPTO support staff.

In the email dated Jun. 25, 2018, I tried to get confirmation of my application status online but was unable to do so. At that time, I called the Application Assistance unit, but I was only able to confirm receipt at the time.

### STATEMENT REGARDING PRIOR DISCLOSURES BY THE INVENTOR OR A JOINT INVENTOR

Not Applicable.

### BACKGROUND OF THE INVENTION

The present invention is in the technical field of PICC, peripherally inserted central catheter, line sleeves. More particularly, the present invention is in the technical field of PICC, peripherally inserted central catheter, line arm sleeves with anti-microbial, odor-resistant, moisture-wicking breathable mesh fabric over the PICC insertion area for survivors undergoing treatment for cancer(s), infectious disease(s), and other medical issues that use PICC lines in the treatment plan.

A PICC line can be used by a patient for an extended period of time. As a result, the PICC line sleeve is worn to cover the catheters caps, which are external to the body. Sleeves help users keep their PICC line insertion area clean and clear from other objects. This present invention gives users a soft, flexible sleeve that holds its shape, provides the ability to view the PICC line insertion area, and supports keeping the area dry and clean.

### BRIEF SUMMARY OF THE INVENTION

The present invention is an arm sleeve designed to provide coverage for peripherally inserted central catheter lines (PICC) lines.

The sleeve is made of mesh fabric to cover the PICC insertion area. The fabric has properties to enhance the quality of everyday use. Key properties of the treated fabric are: anti-bacterial, odor resistant, non-toxic, breathable, fast drying and moisture-wicking. The sleeve may use treated or non-treated solid fabric or treated mesh fabric to in the body of the sleeve. The treated solid fabric has the same properties as the treated mesh fabric.

The sleeve is manufactured in two variations. The variation with the window-style opening design includes fabric in the interior of the sleeve that can be opened and secured while the user is wearing the sleeve. The secured opening in the sleeve is a differentiator for users that allows them to view and access their PICC line without removing their sleeve. This is a unique combination with the treated mesh of the properties stated. The solid fabric used will have a spandex included in the fabric type.

The variation with the slip-on design is made of mesh fabric to cover the PICC insertion area for the circumference of the arm and solid fabric at both ends. The mesh fabric is treated and has properties to enhance the quality of life for everyday use. The solid fabric may be treated or non-treated.

### BRIEF DESCRIPTION OF THE DRAWINGS

FIG. 1 is a schematic drawing of the PICC arm sleeve in the window-style opening variation.

FIG. 1 is a schematic drawing of the PICC arm sleeve in the window-style opening variation with an alternate combination of fabrics.

FIG. 3 is a schematic drawing of the PICC arm sleeve in the slip-on variation.

### DETAILED DESCRIPTION OF THE INVENTION

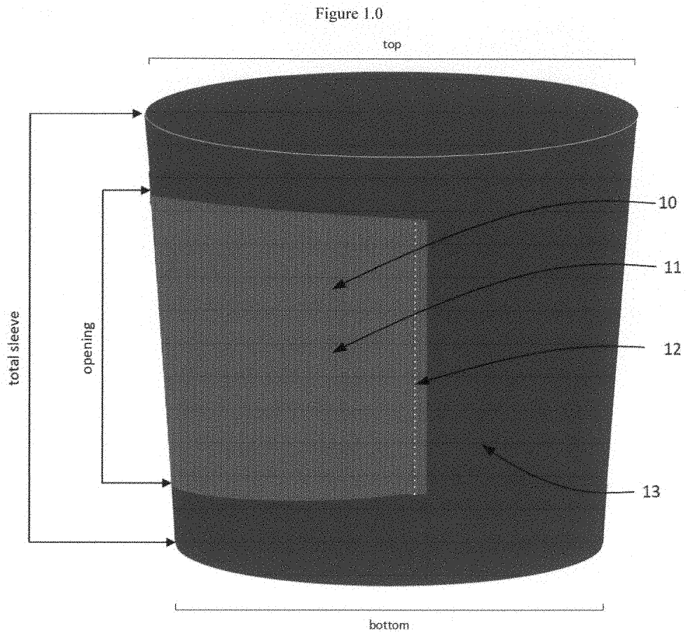

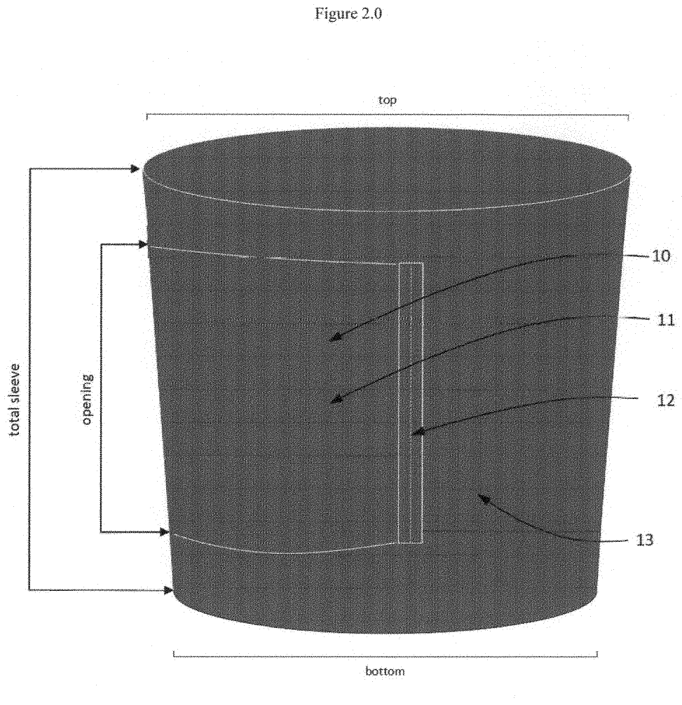

Referring now to the invention in more detail, in FIG. 1.0 the schematic drawing of the sleeve is shown with the window-style opening. The sleeve consists of an opening (10), treated mesh fabric (11), treated fabric lined with a fastener (12), and solid or mesh fabric (13) in the body of the sleeve. The treated mesh fabric (11) has a treatment applied by the textile manufacturer that provides specific attributes to the fabric. Those attributes include anti-bacterial, clean and hygienic, odor resistant, non-toxic, piling resistant, breathable, fast drying, moisture management and wicking, soft hand, and environmentally friendly treatment. The treated mesh fabric (11) will be used in all sleeves, as the mesh will be the fabric covering the PICC insertion area. Treated & non-treated solid or treated mesh fabric (13) may be used in the body of the sleeve. The window opening (10) of the sleeve is made of the treated mesh fabric (11). The long side of the window opening (10) has a fabric strip lined with a fastener (12) that can be separated when pulled apart deliberately. The sleeve also uses a simple brand label to identify the brand of the sleeve and a simple size tag to identify the size of the sleeve.

The design of the sleeve facilitates the user to open and close the window opening (10), while wearing the sleeve. The design uses fabric lined with a fastener (12) to make it easy for the user to open and close the window opening (10) without pulling and tugging on the arm. The properties of the treated fabric, specifically that it is anti-bacterial, moisture-wicking, and breathable accommodate users for wearing the PICC for consecutive hours and while at rest or while active. Additionally, when solid fabric (13) is used, the selected fabric has spandex to allow the sleeve to better maintain its fit on the user.

In FIG. 2.0 the schematic drawing of the sleeve is shown in the window-style opening variation is constructed of all treated mesh fabric in all elements (10), (11), (12), and (13).

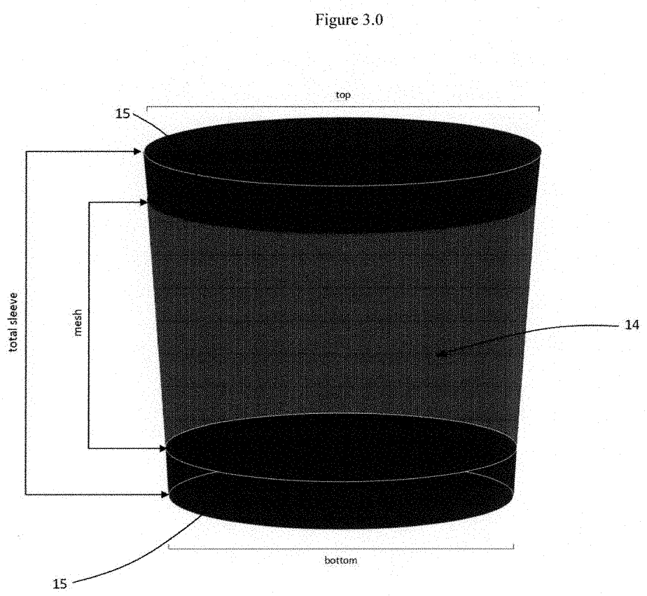

In FIG. 3.0 the schematic drawing is a slip-on design variation of the sleeve with no window opening. The sleeve consists of treated mesh fabric (14) for the full circumference of the arm and solid fabric (15) at the ends. The treated mesh fabric (14) has a treatment applied by the textile manufacturer that provides specific attributes to the fabric. Those attributes include anti-bacterial, clean and hygienic, odor resistant, non-toxic, piling resistant, breathable, fast drying, moisture management and wicking, soft hand, and environmentally friendly treatment. The treated mesh fabric (14) will be used in all sleeves, as the mesh will be the fabric covering the PICC insertion area. Treated & non-treated solid fabric (15) will be used in the sleeve. The treated solid fabric has the same attributes as the treated mesh fabric. The sleeve also uses a simple brand label to identify the brand of the sleeve and a simple size tag to identify the size of the sleeve.

The design of the sleeve as shown in FIG. 3.0 facilitates easy slip-on or slip-off of the sleeve for comfortable, flexible wear and full coverage of the PICC insertion area. The properties of the treated fabric, specifically that it is anti-bacterial, moisture-wicking, and breathable accommodate users for wearing the sleeve for consecutive hours and while at rest or while active. Additionally, the solid fabric (15) used has spandex to allow the sleeve to better maintain its fit on the user.

All variations of the sleeve are constructed by sewing all fabric, notions, and labels together. Where treated or non-treated solid fabric is used for elements (13) or (15), it may vary in style from solids to print colors. Where treated mesh fabric used, it may vary in color.

The present design provides an alternative to leaving the PICC insertion area exposed or using a simple fabric white mesh that may or may not be provided in the hospital. The advantages of the present invention include, without limitation: 1) anti-bacterial, moisture-wicking, odor resistant, and breathable specialty treated mesh fabric 2) treated & non-treated solid fabrics with spandex to provide flexible cover that holds its shape while at rest or while active, where treated solid fabric properties also include being anti-bacterial, moisture-wicking, odor resistant, and breathable; and 3) a convenient window opening, providing flexibility to view & access the PICC line insertion area without removing the sleeve.

In broad embodiment, the present invention is a PICC line arm sleeve with a specialty treated mesh fabric and an opening to cover the PICC insertion area with a fastener along one edge. Use of the sleeve cover is not limited but targeted to survivors undergoing treatment for cancer(s), infectious disease(s), and other medical issues that use PICC lines in the treatment plan.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

1. A garment comprising:
a. pieces of fabric joined together and positioning them together so that a portion of the joined fabrics have an open space to which a separate piece of fabric is joined along one edge of the space and secured to the other fabric on the opposite side of the space with a fastener that can be separated when pulled apart deliberately, and
b. said separate piece of fabric has an antibacterial treatment applied,
whereby altogether said fabrics protect an external area on the body.

2. A garment comprising pieces of fabric joined together and positioning them together so that a portion of the joined fabrics have antibacterial treatment applied whereby said fabrics protect an external area on the body.
